(19) United States
(12) Patent Application Publication
(10) Pub. No.: US 2007/0059296 A1
(43) Pub. Date: Mar. 15, 2007

(54) Probiotic composition having acid-resistant enteric coating

(76) Inventor: Liang-Jung Chen, Jhunan Township (TW)

Correspondence Address: J.C. Patents, 4 Venture, Suite 250, Irvine, CA, 92618, US

(21) Appl. No.: 11/471,248

(22) Filed: Jun. 19, 2006

(30) Foreign Application Priority Data
Sep. 13, 2005 (TW) 094131403

(51) Int. Cl. A61K 35/74; A61K 31/717

(52) U.S. Cl. 424/93.45; 514/57

(57) ABSTRACT

A probiotic composition essentially comprises 15 to 20 wt % of milk powder, 25 to 30 wt % of corn starch, 8 to 15 wt % of modified starch (capsul), 10 to 15 wt % of ethylcellulose, 5 to 15 wt % of bacterial broth, and 10 to 15 wt % of talc. The probiotic composition is microencapsulated to form a plurality of microencapsule coated with an acid-resistant enteric coating for improving the enteric acid-resistance, the probiotic survival rate, the antimicrobial property, the stability, the moisture-proof property, and the mobility of the probiotic composition preventing from coagulation in a moist environment and for being used as an additive applied to livestock feed.

### CROSS-REFERENCE TO RELATED APPLICATION

This application claims priority benefit of Taiwan application No. 094131403 filed on Sep. 13, 2005, the contents of which is hereby incorporated by reference in its entirety.

### BACKGROUND OF THE INVENTION

1. Field of the Invention

The present invention relates to a probiotic composition having an acid-resistant enteric coating. More particularly, the present invention relates to a microencapsulated probiotic composition having an acid-resistant enteric coating for improving the enteric acid-resistance, the probiotic survival rate, and the effectiveness of the probiotic composition.

2. Description of the Related Art

Conventionally, most of the feed additives of economic animals essentially comprise at least one kind of antibiotic, but the abuse of the antibiotic induces the drug resistance of the related pathogens so that the use of the antibiotic loses its efficacy against the related pathogens and can not reduce the prevalence rate of the related infectious disease. In another aspect, after a person ate the food containing antibiotic residues, levels of antibiotic had further accumulated within the body of the person resulting in greater health risk. For example, the overdose of tetracycline causes the deficiency or retardation of osteo-calcification, and the failure of some first-line antibiotics increases the prevalence rate of some infectious diseases such as tuberculosis. In addition, excess of cholesterol and triglyceride of livestock, such as pigs, is not good for health of consumers, especially related to obesity and cardiovascular diseases. Presently, native and foreign related research institutes consider the problem about the abuse of various antibiotics so as to suggest the use of non-medicinal feed additives in substitution for antibiotics. Among the non-medicinal feed additives, probiotic bacteria are considered as an agent for facilitating the digestion of the digestive systems of host animals.

The present invention intends to provide a probiotic composition having an acid-resistant enteric coating in such a way as to mitigate and overcome the above problem. Furthermore, the probiotic composition having the acid-resistant enteric coating is microencapsulated. Accordingly, this ensures improvements of the enteric acid-resistance, the probiotic survival rate, and the effectiveness of the probiotic composition.

### SUMMARY OF THE INVENTION

The primary objective of this invention is to provide a probiotic composition having an acid-resistant enteric coating, wherein the probiotic composition essentially comprises: (a) 15 to 20 wt % of milk powder, (b) 25 to 30 wt % of corn starch, (c) 8 to 15 wt % of modified starch (capsul), (d) 10 to 15 wt % of ethylcellulose, (e) 5 to 15 wt % of bacterial broth, and (f) 10 to 15 wt % of talc. Probiotic bacteria of the bacteria broth of the probiotic composition is coated with the acid-resistant enteric coating and dried by spray drying under 65° C. for removing water so as to be microencapsulated to form a plurality of microencapsule. After feeding livestock with the microencapsulated probiotic composition, the probiotic bacteria proliferate in the gastrointestinal tract of the livestock while inhibiting pathogenic microorganisms in the gastrointestinal tract. Meanwhile, the probiotic bacteria facilitate feed digestion for increasing digestibility, synthesize various vitamins improving the resistance against diseases, and accelerating the growth of the livestock.

The secondary objective of this invention is to provide a probiotic composition having an acid-resistant enteric coating, wherein based on conventional enteric-coated microencapsulation method is only applied to the manufacture of human medicines but not to the manufacture of livestock feed additives or medicines in Taiwan or other regions, the present invention selects suitable species of probiotic bacteria that are coated with the acid-resistant enteric coating and dried by spray drying so as to be microencapsulated to form a plurality of microencapsule. Meanwhile, the present invention adjusts the microencapsulated probiotic composition for increasing intervals between the microencapsule adjacent to each other so as to improve the released rate of the probiotic bacteria from the microencapsule.

Further scope of the applicability of the present invention will become apparent from the detailed description given hereinafter. However, it should be understood that the detailed description and specific examples, while indicating preferred embodiments of the invention, are given by way of illustration only, since variation will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus are not limitative of the present invention, and wherein:

FIG. 1 is a schematic view illustrating microencapsule of a probiotic composition having an acid-resistant enteric coating, which are passed through a gastric acid environment, in accordance with a preferred embodiment of the present invention;

FIG. 2 is a micrograph of 5000×SEM (scanning electron microscope) showing the tight structure of the microencapsule in an acidic solution (pH 1.5) in accordance with the preferred embodiment of the present invention; and

FIG. 3 is a micrograph of 5000×SEM showing the degraded structure of the microencapsule in a neutral solution (pH 7.4) in accordance with the preferred embodiment of the present invention.

### DETAILED DESCRIPTION OF THE INVENTION

The present invention is related to a probiotic composition which essentially comprises 15 to 20% by weight of milk powder, 25 to 30% by weight of corn starch, 8 to 15% by weight of modified starch (capsul), 10 to 15% by weight of ethylcellulose, 5 to 15% by weight of bacterial broth, and 10 to 15% by weight of talc is coated with an acid-resistant enteric coating. Referring now to Table 1, eight experimental groups of the probiotic composition in a microencapsulated form according to the preferred embodiment of the present invention is shown. In preparation, preparing 1 unit volume of the bacterial broth containing probiotic bacteria PS551, and then preparing 10 unit volume of MRS medium (Man Rogosa Sharpa) to mix with the bacterial broth. The mixture of the bacterial broth and the MRS medium is poured into a 550 ml flask for cultivating about 16 hours under 37° C. The enteric coating comprises at least one modified derivative of methylcellulose, and the probiotic bacteria are preferably selected from Lactobacillus. The enteric coating of the probiotic composition of the preferred embodiment of the present invention in the Table 1 can be classified into two major types: the first type mainly comprises the modified starch (capsul) ingredient, and the second type mainly comprises the ethylcellulose ingredient. In microencapsulated processing, the bacterial broth having the probiotic bacteria is mixed with the enteric coating having water-solubility and at least one kind of excipient, and then stirs the mixture thereof. After stirring, the mixture is processed by co-spray drying to form a plurality of microencapsule in a powder form.

TABLE 1

| | | | modified | | | |
|---|---|---|---|---|---|---|
| | milk | corn | starch | ethyl | bacterial | |
| | powder | starch | (capsul) | cellulose | broth | talc |
| group | (g) | (g) | (g) | (g) | (g) | (g) |
| EC-0 | 3 | 20 | 10 | 0 | 400 | 8 |
| EC-0.5 | 3 | 20 | 10 | 2 | 400 | 8 |
| EC-1 | 3 | 20 | 10 | 4 | 400 | 8 |
| EC-2 | 3 | 20 | 10 | 8 | 400 | 8 |
| EC-4 | 3 | 20 | 10 | 16 | 400 | 8 |
| EC-41 | 3 | 20 | 0 | 16 | 400 | 8 |
| EC-42 | 3 | 20 | 0 | 32 | 400 | 8 |
| EC-43 | 3 | 20 | 0 | 48 | 400 | 8 |

The enteric coating of the preferred embodiment of the present invention is acid-resistant against an acidic solution such as gastric acid so that the probiotic bacteria coated by the enteric coating are not in contact with gastric acid and/or gastric enzymes in the stomach of host animals. When the microencapsule of the probiotic composition are approached the small intestine, the enteric coating of the microencapsule is immediately dissolved and degraded within the small intestine (pH 6.8-7) while a portion of the probiotic composition is still not dissolved and degraded for maintaining a spherical frame of the microencapsule so that the probiotic bacteria can be released from crevices of the spherical frame of the microencapsule. It can be understood that the macromolecular material of the enteric coating covers small particles of the probiotic composition which has a particle size ranged from nanometers to micrometers. Based on the self-adjustability of the thickness, the hardness, and the solubility of the enteric coating, the microencapsule are capable of releasing a suitable amount of the probiotic bacteria at a suitable time and/or in a suitable position of the gastrointestinal tract.

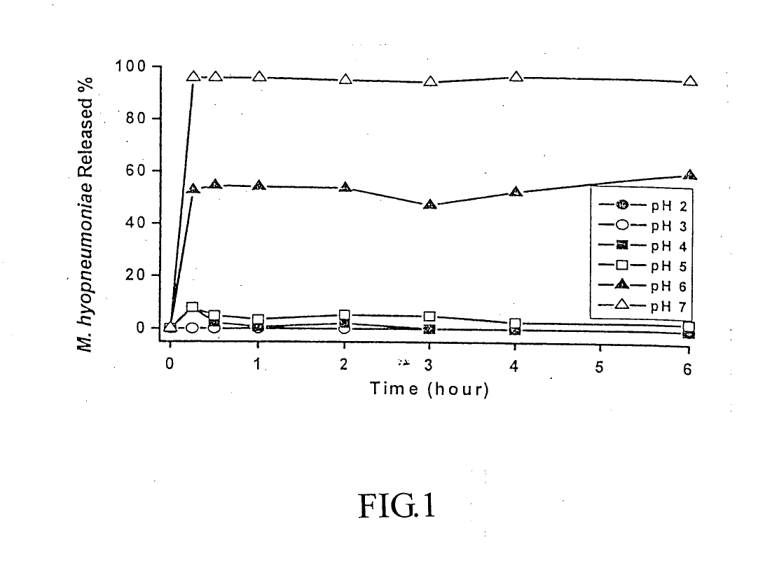

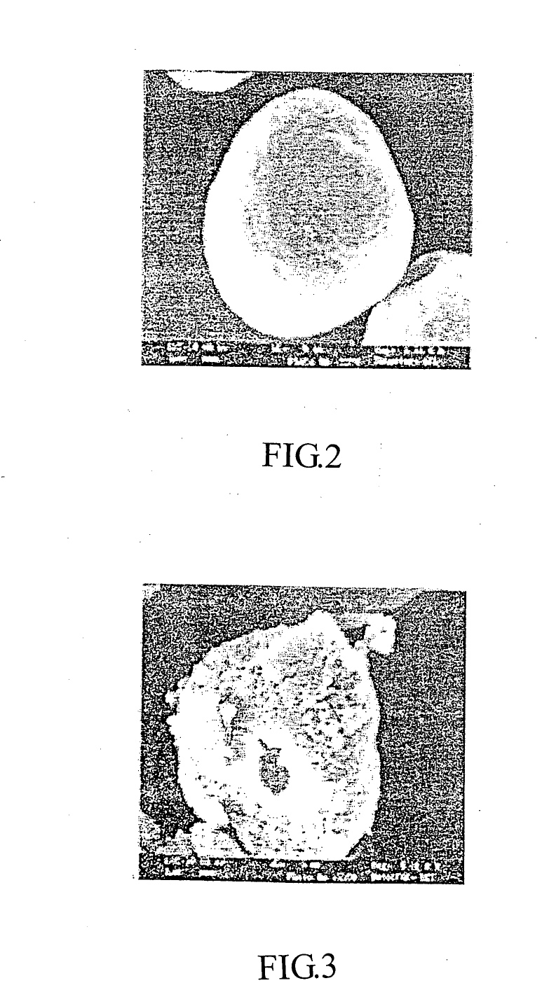

Referring now to FIG. 1, a schematic view of the microencapsule of the probiotic composition having the acid-resistant enteric coating, which are passed through a gastric acid environment, in accordance with a preferred embodiment of the present invention is illustrated. The microencapsule of the probiotic composition are degraded to different degrees in different pH environments. Particularly, in a gastrointestinal environment of pH 6.8˜7, the released rate of Mycoplasma hyopneumoniae is increased in proportion to time. Referring now to FIGS. 2 and 3, micrographs of 5000×SEM (scanning electron microscope) of the structures of the microencapsule in different positions in a gastrointestinal tract of a host animal are shown. The degradation of the microencapsule is observed by the 5000×SEM (scanning electron microscope). Referring to FIG. 2, the tight and rigid structure of the microencapsule can be observed in an acidic solution (pH 1.5). Referring to FIG. 3, the degraded and dissolved structure of the microencapsule can be observed in a neutral solution (pH 7.4). Thereby, it was the evidence of the enteric solubility of the microencapsule of the probiotic composition in accordance with the preferred embodiment of the present invention.

The invention will now be further explained and illustrated by reference to the following non-limiting examples.

### EXAMPLE 1

In preparation, preparing the bacterial broth containing the probiotic bacteria, the enteric coating having water-solubility and acid-resistant property, and the excipient. Then, the bacterial broth is mixed with the enteric coating and the excipient. The mixture is processed by co-spray drying to form a plurality of microencapsule in a powder form. After dried, sampling the microencapsule for measuring counts of the probiotic bacteria thereof. Referring now to Table 2, the coating properties of the microencapsule having different enteric coatings of eight experimental groups of probiotic compositions according to Table 2 of the preferred embodiment of the present invention are listed. The EC-0 group as shown in Table 2 has an average coating rate ranged from (80±2)% to (81±4)% when the probiotic bacteria are selected from PS551 or L103. The probiotic survival rate of the L103 in an endospore form is 96%, approximately about 100%. The probiotic survival rate of the PS551 is 67%. The EC-0.5 group as shown in Table 1 and 2 further contains ethylcellulose (ECN-7) about 0.5%, and the probiotic survival rate of the PS551 is approximately about 92%. The more the ethylcellulose (ECN-7) ingredient is added, the higher the probiotic survival rate of the PS551 or the L103 was. Referring to Table 2, the EC-2 group contains ethylcellulose (ECN-7) about 2%, and the probiotic survival rate of the PS551 and L103 is approximately about 100%.

TABLE 2

| Experimental groups of | | | | | | |
|---|---|---|---|---|---|---|
| the probiotic compositions | | | | Coating properties | | |
| | modified starch | ethylcellulose | | Average coating | counts of probiotic | probiotic |
| I.D | (capsul) (g) | (g) | (%) | rate (%) | bacteria (cfu/g) | survival rate (%) |
| EC-0 | 10 | 0 | 0 | PS551 81 ± 4 | 9.4 ± 0.2 × 10¹⁰ | 67 |
| | | | | L103 80 ± 2 | 3.1 ± 0.4 × 10⁹ | 96 |
| EC-0.5 | 10 | 2 | 0.5 | PS551 90 ± 3 | 1.3 ± 0.2 × 10¹¹ | 92 |
| | | | | L103 90 ± 2 | 3.1 ± 0.4 × 10⁹ | 98 |
| EC-1 | 10 | 4 | 1 | PS551 93 ± 5 | 1.3 ± 0.2 × 10¹¹ | 96 |
| | | | | L103 92 ± 2 | 3.1 ± 0.4 × 10⁹ | 97 |
| EC-2 | 10 | 8 | 2 | PS551 100 ± 6 | 1.4 ± 0.4 × 10¹¹ | 100 |
| | | | | L103 96 ± 2 | 3.2 ± 0.2 × 10⁹ | 100 |
| EC-4 | 10 | 16 | 4 | PS551 98 ± 2 | 1.3 ± 0.4 × 10¹¹ | 98 |
| | | | | L103 92 ± 2 | 3.2 ± 0.3 × 10⁹ | 100 |
| EC-41 | 0 | 16 | 4 | PS551 98 ± 5 | 1.1 ± 0.3 × 10¹¹ | 76 |
| | | | | L103 92 ± 2 | 3.1 ± 0.4 × 10⁹ | 98 |
| EC-42 | 0 | 32 | 8 | PS551 93 ± 3 | 9.3 ± 0.6 × 10¹⁰ | 65 |
| | | | | L103 92 ± 2 | 3.1 ± 0.4 × 10⁹ | 98 |
| EC-43 | 0 | 48 | 12 | PS551 71 ± 3 | 8.1 ± 0.5 × 10¹⁰ | 57 |
| | | | | L103 80 ± 2 | 3.0 ± 0.4 × 10⁹ | 94 |

Ps. cfu/g is colony forming unit per gram.

### EXAMPLE 2

As described in Example 1, the probiotic composition is coated with the enteric coating, and process by co-spray drying to form a plurality of microencapsule in a powder form. The probiotic composition having the probiotic bacteria PS551 is microencapsulated and evaluated its acid-resistance against gastric acid (HCl) as shown in Table 3. In a dissolution experiment, it was found that the EC-0, EC-0.5, EC-1, EC-2 and EC-41 groups of the probiotic compositions as shown in Table 3 have no acid-resistance. Although the enteric coatings of the EC-0, EC-0.5, EC-1, and EC-2 groups of the probiotic compositions are not degraded or dissolved in an acidic solution, the acidic solution (0.03N HCL) can penetrate through the enteric coatings to be in contact with the probiotic bacteria (PS551) so as to reduce the probiotic survival rate thereof (only about 0.02%-0.3%). In a preferred embodiment, the present invention further contains an antacid material (economic available product: Magaldrate) about 4 g in the EC-4M and the EC-41M groups as shown in Table 3. The probiotic survival rate of the probiotic bacteria is approximately from 50% to about 99% under 0.01N HCl after 1 hour while the probiotic survival rate of the probiotic bacteria is approximately from 8% to about 33% under 0.03N HCl after 1 hour.

TABLE 3

| | | | | Counts and probiotic survival rate of | | | |
|---|---|---|---|---|---|---|---|
| 400 ml probiotic bacteria broth (PS551) | | | | probiotic bacteria of the microencapsule | | | |
| mixed with three ingredients | | | | under acidic environments (HCl) | | | |
| | modified | ethyl | antacid | under 0.03N HCl | | under 0.01N HCl | |
| | starch (capsul) | cellulose | material M | counts | rate | counts | rate |
| I.D | (g) | (g) | (g) | cfu/g | (%) | cfu/g | (%) |
| EC-0 | 10 | 0 | 0 | 2.4 ± 0.2 × 10⁶ | 0.02 | — | — |
| EC-0.5 | 10 | 2 | 0 | 4.8 ± 0.2 × 10⁷ | 0.03 | — | — |
| EC-1 | 10 | 4 | 0 | 3.6 ± 0.2 × 10⁸ | 0.3 | — | — |
| EC-2 | 10 | 8 | 0 | 5.4 ± 0.4 × 10⁸ | 0.4 | 7.5 ± 0.4 × 10⁹ | 8 |
| EC-4 | 10 | 16 | 0 | 7.2 ± 0.3 × 10⁸ | 0.7 | 1.4 ± 0.3 × 10¹⁰ | 10 |
| EC-4M | 10 | 16 | 4 | 4.6 ± 0.1 × 10¹⁰ | 33 | 1.4 ± 0.2 × 10¹¹ | 99 |
| EC-41 | 0 | 16 | 0 | 2.3 ± 0.1 × 10⁸ | 0.2 | 3.6 ± 0.1 × 10⁹ | 3 |
| EC-41M | 0 | 16 | 4 | 1.1 ± 0.3 × 10¹⁰ | 8 | 7.3 ± 0.4 × 10¹⁰ | 50 |

### COMPARATIVE EXAMPLE 1

Conventional probiotic composition products having Lactobacillus are selected from (a) Yoca feed additive (Lactozyme Enterprise Co., Ltd.; Taiwan) having 8 species of Lactobacillus and 6 types of digestive enzymes; (b) LBC feed additive (Cerbios-Pharma S.A.; Switzerland). The Yoca and LBC feed additive are compared with (c) the microencapsulated probiotic composition (EC-4M group) having the probiotic bacteria PS551 as shown in Table 3 according to the preferred embodiment of the present invention. The counts of the probiotic bacteria before/after acid treatment (HCl) of the Yoca feed additive, the LBC feed additive, and the microencapsulated probiotic composition of the present invention are listed in Table 4 for assessing the acid-resistance thereof. As the result in Table 4, the counts of the probiotic bacteria of the Yoca and LBC feed additive are met their product specifications, but the Yoca and LBC feed additive almost have no acid-resistance. Although the Yoca feed additive contains an antacid material, the diluted solution of the Yoca feed additive still loses its acid-resistance. Due to the Yoca and LBC feed additive have no any acid-resistant enteric coating, the probiotic survival rate thereof is very low, and the counts of the probiotic bacteria are substantially lower than 100 under 0.03N HCl after 2 hours. The microencapsulated probiotic composition of the present invention has more counts of the probiotic bacteria (4.6×10) and relatively higher probiotic survival rate (33%) due to the acid-resistant enteric coating.

TABLE 4

| | | cfu/g (%) | cfu/g (%) |
|---|---|---|---|
| | | 0.03N HCl | 0.03N HCl |
| Sample | cfu/g | after 15 min | after 2 hrs |
| (a) Yoca | 1.5 × 10⁹ | 1.5 × 10⁹ (0.008%) | <10²(0.000%) |
| (b) LBC | 1.6 × 10⁹ | 1.5 × 10⁹ (0.008%) | <10²(0.000%) |
| (c) PS551 EC-4M | 1.4 × 10¹¹ | — | 4.6 × 10¹⁰(33%) |

Result

As described above, referring back to Table 1 and 2, the probiotic composition in accordance with the preferred embodiment of the present invention can increase the probiotic survival rate of the probiotic bacteria of the microencapsule having the enteric coating by increasing the rate of ethylcellulose. Furthermore, referring back to Table 1 and 3, the probiotic composition in accordance with the preferred embodiment of the present invention can increase the acid-resistance against gastric acid of the probiotic bacteria (PS551) of the microencapsule having the enteric coating by adding the antacid material.

Although the invention has been described in detail with reference to its presently preferred embodiment, it will be understood by one of ordinary skill in the art that various modifications can be made without departing from the spirit and the scope of the invention, as set forth in the appended claims.

What is claimed is:

1. A probiotic composition, comprising: 15 to 20% by weight of milk powder, 25 to 30% by weight of corn starch, 8 to 15% by weight of modified starch (capsul), 10 to 15% by weight of ethyl cellulose, 5 to 15% by weight of bacterial broth, and 10 to 15% by weight of talc, the probiotic composition coated with an acid-resistant enteric coating and mixed with at least one excipient, the mixture of the probiotic composition, the acid-resistant enteric coating, and the excipient is co-spray dried.

2. A probiotic composition as defined in claim 1, wherein the bacterial broth comprises at least one species of probiotic bacteria selected from Lactobacillus.

3. A probiotic composition as defined in claim 1, wherein the enteric coating is selected from at least one modified derivative of methylcellulose.

4. A probiotic composition as defined in claim 1, wherein the mixture of the probiotic composition, the acid-resistant enteric coating, and the excipient is co-spray dried to form a plurality of microencapsule in a powder form.

5. A probiotic composition as defined in claim 1, wherein the probiotic composition further comprises an antacid material for increasing the probiotic survival rate of at least one species of probiotic bacteria within the probiotic broth.

6. A probiotic composition as defined in claim 1, wherein the bacterial broth comprises at least one species of probiotic bacteria selected from Enterococcus.
